(19) United States
(12) Patent Application Publication
(10) Pub. No.: US 2008/0208295 A1
(43) Pub. Date: Aug. 28, 2008

(54) Phototherapy Treatment and Device to Improve the Appearance of Nails and skin

(76) Inventors: William E. Cumbie, Yorktown, VA (US); Douglas B. Juanarena, Blacksburg, VA (US)

Correspondence Address: MEREK, BLACKMON & VOORHEES, LLC, 673 S. WASHINGTON ST., ALEXANDRIA, VA, 22314, US

(21) Appl. No.: 12/039,006

(22) Filed: Feb. 28, 2008

Related U.S. Application Data
(60) Provisional application No. 60/891,972, filed on Feb. 28, 2007.

(51) Int. Cl. A61B 18/18

(52) U.S. Cl. 607/88

(57) ABSTRACT

A method and apparatus for using phototherapy to beneficially improve the appearance of nails and skin. Light can be applied to nails and skin that is primarily germicidal to inactivate organisms that cause infections; applied as primarily cleansing light in which case it primarily improves the appearance of the nails and skin; or applied as a combination of the two, namely germicidal and cleansing light, in which case it is termed cleansing light. By applying the light for a set time and intensity to nails can be improved in appearance, whether or not an infection is present. By applying the light to skin with or without the presence of other compounds, skin appearance can be approved.

### BACKGROUND—FIELD OF INVENTION

This invention relates to the use of phototherapy to improve the appearance of nails and skin.

### BACKGROUND OF THE INVENTION—OBJECTS AND ADVANTAGES

Phototherapy has recently been used to treat infected nails using germicidal light. However, it appears that phototherapy can also be used to improve the appearance beyond what is attributable to inactivation of infection causing organisms alone. The advantage of using phototherapy to improve the appearance of nails instead of or in addition to using it to kill or inactivate infection causing organisms is that the results of such treatment can be seen much earlier. Thus a person using a phototherapy treatment to improve the appearance of nails may see results more quickly than one receiving a treatment that works solely by inactivation of the organisms that cause infections. In addition, this technology can be extended to improving the appearance of skin in a similar manner.

### SUMMARY OF INVENTION

Light has been shown to help improve the appearance of skin by removing wrinkles and producing tans. Sunlamps are another variety of devices that are used to condition skin so that it appears better, that is so that it appears healthier, more aesthetically pleasing, or the like. Sunlamps are rich in UV and act to increase or release of the pigment melanin into the skin's cells after exposure to ultraviolet radiation, thus making the skin browner, which in many people's opinion enhances its appearance.

However, light has never been used to improve the appearance of nails. Trying to use light to improve the appearance of nail has never been tried possibly due to the fact that nails are composed of dead keratin and it might have been thought that this could not be conditioned easily. However, it is also true that hair is composed of dead keratin which can be conditioned to change its appearance (which happens when people lay out in the sun for a long period of time making their hair appear lighter). Thus, while it is unobvious and novel that light can be used to improve the appearance of nails, it is not impossible that this is so.

The invention is a method and device to condition nails with light in a manner that improves their appearance. While the improvement may be associated with the inactivation of certain organisms, it can also be due to the beneficial conditioning caused by light when applied to the nails themselves.

Additionally, this invention can be used to improve the appearance of skin by lightening and/or whitening it. Thus it can be used in a manner similar to chemical bleaching agents, such as 2% hydroquinone used in some countries. Additionally, the light can be used alone or it can be used in conjunction with bleaching agents for an effect that is better than using the bleaching agent by itself

### BRIEF DESCRIPTION OF DRAWINGS

FIG. 1 is a graph showing the spectrum of xenon pulsed light (designated Prototype B) used for clinical trials. This spectrum represents what is sometimes termed ‘low current’ operation of a xenon pulsed light.

FIG. 2 is a graph showing the spectrum of another xenon pulsed light being used in experiments. This spectrum represents what is sometimes termed ‘high current’ operation of a xenon pulsed light.

FIG. 3 is a graph showing a comparison of the light spectra for high (hi) and low (lo) current operation of a light.

### DETAILED DESCRIPTION OF INVENTION

Light can be applied to the nails to condition them and improve their appearance. In a clinical trial it has been show that both monochromatic UVC light and broad spectrum light can improve the appearance of the nails. Moreover, although some of the improvement may be due to the light's ability to inactivate organisms, the light itself can also condition the nail to beneficially improve its appearance. This is evident since several nails showed improvement of appearance after only one treatment and one week of elapsed time after that treatment. This was too early to have the infected part of the nail totally grow out; therefore, in addition to the light inactivating the organism it can beneficially alter the appearance of the nail.

Similarly, cleansing light can be used to improve the appearance of skin by lightening it or in other beneficial ways. This light can be used in conjunction with topical applications to further improve its efficacy.

Clinical Trials Show that Phototherapy Can Be Used to Improve the Appearance of Nails

Clinical trials were conducted using two prototype devices, each of which was used to treat 15 subjects each (30 total). One device, labeled Prototype A, was a low pressure mercury lamp (LPML) that was capable of providing at total of 9 mw/cm2 of UVC at 254 nm over the area to be treated. The second device labeled Prototype B, was a pulsed xenon lamp (XPL) capable of providing pulsed full spectrum light from approximately 180 nm to more than 1600 nm. Prototype B was capable of providing a approximately 9 mw/cm2 of UVC from 240 to 280 nm over the area to be treated.

Fifteen subjects received treatment using Prototype A. Each treatment irradiated the target nail with 17 mw/cm2 of UVC at 254 nm for 45 minutes for 4 sessions each spaced one week apart. Total dosage of UVC to the top of the nail was therefore approximately 22 J/cm2. UVC light was generated by a commercially available low pressure mercury lamp manufactured by Heraeus (model NG6062) which is used to disinfect surfaces such as food containers.

Fifteen subjects received treatment using Prototype B. Each treatment irradiated the target nail with 9 mw/cm2 of UVC from 240 to 280 nm for 8 minutes for 4 sessions each spaced one week apart. Total dosage of UVC to the nail was therefore approximately 4.4 J/cm2. Light for treatment was generated by a commercially available xenon pulsed lamp manufactured by Xenon (model RC-700) which is used to disinfect surfaces such as food containers. In addition to the UVC provided, each treatment included applying the following dosages of light for eight minutes:
Far UV from 180 nm to 240 nm—1.8 J/cm²
UC from 240 nm to 280 nm—4.4 J/cm
UVB from 280 nm to 320 nm—8.7 J/cm
UVA from 320 nm to 400 nm—34.5 J/cm²
Visible light from 400 nm to 750 nm—260 J/cm²
Infrared from 750 nm to 880 nm—1 J/cm²
Infrared above 880 nm—estimated to be about 60 J/cm²

Note that total dosage of light in the UV range was approximately 50 J/cm2 and total overall light dosage (between 180 and 880 nm) was 310 J/cm2 for each of the treatments with the xenon pulsed light. These values have been adjusted to account for use of the SED240 detector with NS254 filter and a 2 order of magnitude neutral density filter that was used to calculate the amount of UVC produced. Other spectrum values were calculated using the UVC reading as the base reading. It should be noted that the light has emissions up through 2600 nm and the light in this extended spectrum may also contribute synergistically to treatment.

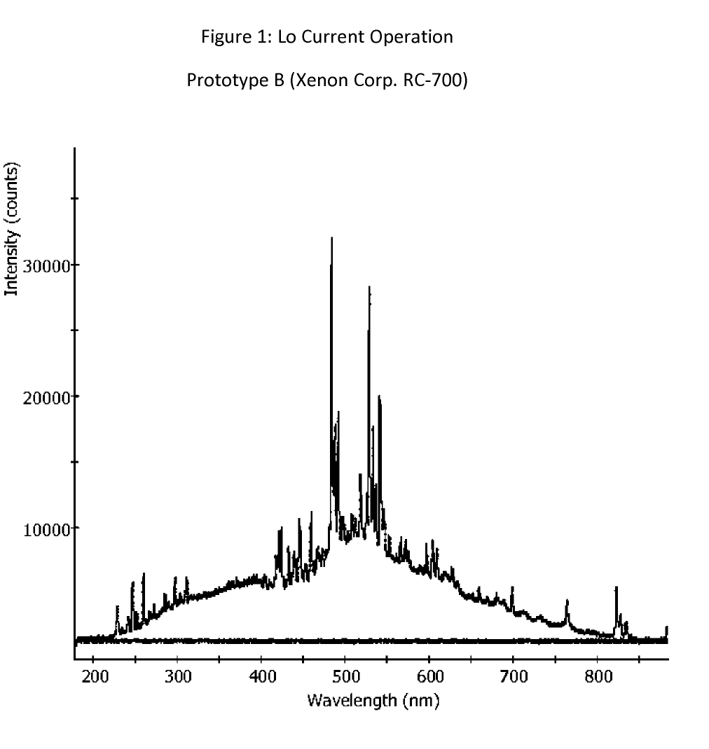

A spectrum of this light is attached as FIG. 1.

Results of Clinical Trials—Of the 15 subjects treated with Prototype A, three dropped out of the study and 12 were evaluated after 1, 2, 3, 8, and 12 weeks. Average clear new nail growth was 2.5 mm after 12 weeks with 7 of the 12 subjects having more than 2 mm of new nail growth. Subjects were also graded on the IGA (Investigator Global Assessment) scale of 0 to 5 for nail appearance. On average there was a 0.27 improvement on this scale (changing from 2.64 to 2.37). Improvement in the IGA score was noted in one nails after 2 treatments and 3 weeks had elapsed (3 weeks from the firs treatment) and one nail after 3 treatments and 4 weeks had elapsed.

Of the 15 subjects treated with Prototype B, two dropped out of the study and 13 were evaluated after 1, 2, 3, 8, and 12 weeks. Average clear new nail growth was 2.5 mm after 12 weeks with 8 of the 13 subjects having more than 2 mm of new nail growth. Subjects were also graded on the IGA (Investigator Global Assessment) scale of 0 to 5 for nail appearance. On average there was a 0.69 improvement on this scale (changing from 2.64 to 2.35). Additionally, it was noted that 2 of the 13 nails showed 1 IGA improvement after only 1 treatment with the assessment being performed 1 week after the first treatment.

The data indicate that both devices were capable of efficaciously treating the nail for infection to so that new nail growth would be clear. Additionally, however, it was noted that the broad spectrum xenon pulsed light was also capable of providing rapid improvement in the appearance of the nail and that in some cases this improvement occurred very quickly. This is a very surprising and unobvious result since treatments for infected nails that rely on inactivating the organisms that cause infections take a longer time before it is possible to see improvement in the appearance of nails.

It appears, therefore, that UVC applied to nails is able to stimulate the growth of clear new nails in nails that are not totally clear. It also appears that in addition to stimulating new nail growth, pulsed broad spectrum light is able to improve the appearance of the nail being treated. While light treatment with UVC alone improved the appearance of the nail somewhat, it did not improve it nearly as much as the xenon pulsed light treatment. Additionally, the improvement in the appearance of nails occurred much more quickly with the xenon pulsed light than with the UVC light treatment. Thus, while treatment with UVC alone can improve the appearance of the nails, treatment with broad spectrum light can improve the appearance of the nail more and it can do so more quickly.

### How Phototherapy Improves the Appearance of Nails and Skin

Phototherapy can improve the appearance of nails in two ways. First, it can improve the appearance of an infected nail by inactivating organisms that cause nail infections. This can be termed the improvement of the appearance of nails using germicidal light. Second, it can improve the appearance of a nail (infected or non-infected) but conditioning the nail and any material that may be associated with it (such a debris, organisms, pigments, etc.). This may be termed the improvement of the appearance of nails using conditioning light. Use of both type of light together can be termed cleansing light. Germicidal, conditioning, and cleansing light can similarly be used to improve the appearance of skin.

### Improving the Appearance of Nails and Skin Using Germicidal Light

If a nail is infected its appearance can often be improved by the application of germicidal light to inactivate the organisms causing the infection. Different wavelengths of light have different germicidal properties. The most germicidal bandwidth of light is the UVC band between 240 nm and 280 nm. This wavelength inactivates organisms primarily by damaging the genetic material of the organism and preventing it from reproducing. UVB has some germicidal properties but it is estimated to be between three to six orders of magnitude less effective ( 1/1,000th to 1/1,000,000th). Other bands of light are generally considered less germicidal. Combinations of various bands of light may be more germicidal, especially if they contain a component of UVC. Thus nails can be improved using germicidal light as it inactivates the organisms causing infections thus permitting the nail to grow out clearly. Skin can similarly be improved using germicidal light.

### Improving the Appearance of Nails and Skin Using Conditioning Light

Nails can also be improved by the use of conditioning light that alters the composition of the nail and any material that may be associated with it, such as debris, organisms, pigments, etc. Conditioning light can alter the composition of the nail in a number of ways such as by breaking molecular bonds. Breaking of such bonds is similar to the use of sunlight to bleach hair. When sunlight bleaches hair, it breaks the bonds of chromophores. Chromophores are molecules that are capable of absorbing certain wavelengths of light thus making the molecule appear to be a certain color. When the bonds of chromophores are broken then they lose the ability to absorb the specific wavelength of light that they did formerly. Thus, when sunlight strikes the chromophores of hair it breaks the molecule into two smaller fragments and each of the smaller fragments are less able to absorb light thus the hair appears to have less color (i.e. it is more white which many call blond or bleached). Conditioning light that break the bonds of chromophores is generally associated with more energetic light—in the case of the bleaching of hair it is the UVB and UVA bandwidth that is most bleaching. The above example of how light can condition nails is illustrative and not exhaustive. Those skilled in the art will recognize other ways that light can be used to condition nails beneficially. It should also be noted that the chromophores that are affected may reside in organisms and debris associated with the nails, debris produced by the organisms, as well as chromophores in the nails themselves. Thus, black chromophores from debris or organisms that resides beneath the nail may be ‘bleached’ by phototherapy treatment such that the appearance of the nail is improved.

Additionally, it should be noted that it is not only the bandwidth of light that is a characteristic of the light that determines its properties. Effective conditioning light characteristics are determined by dosage applied. The intensity and modulation of conditioning light will also affect its ability to improve the appearance of nails. For example, pulsing of conditioning light permits extremely high instantaneous peaks of light that can quickly break the chemical bonds of chromophores. The pulsing of light also permits the application of very high peaks of light while limiting the overall dosage of light applied since pulsed light is generally ‘off’ for far longer periods that it is ‘on’. Thus, the use of pulsed light with high instantaneous peaks is a preferred manner of conditioning nails.

It should also be noted that conditioning light may improve the appearance of a nail, even if it is not found to be infected, since its action does not rely on the light being merely germicidal. Thus a person with a yellow nail that did not appear to be affected could use conditioning light to improve its appearance.

One of our discoveries is that nails too can be treated using conditioning light so that their appearance can be improved even if they are not infected. While not wishing to be bound by specific wavelengths it appears that a substantial amount of conditioning light that can improve the appearance of nails is primarily in the UVA and UVB range of 280 nm to 400 nm but that this also extends to the visible bands and infrared bands.

Additionally, the use of broad band light can be particularly effective as a conditioning light since it contains the wavelengths of all the visible bands of light. This light can break the bonds of chromophores that are in the visible range thus changing these chromophores so that the nails have a better visual appearance. The wide spectrum light also penetrates differentially to various depths depending on the specific wavelength of each photon which thus permits the entire nail to be treated.

It is also possible to improve the appearance of skin using conditioning light as outlined above.

### Improving the Appearance of Nails and Skin Using Cleansing Light

As noted in the previous sections concerning germicidal and conditioning light, the bandwidths of these types of lights are somewhat overlapping. While germicidal light is primarily in the UVC range (and to a lesser extent the UVB range), conditioning light is primarily in the UVA range (but can extent to the other parts of the UV spectrum and the visible light spectrum). It is possible to combine these two types of light synergistically to form what is called cleansing light. This type of light can beneficially improve the appearance of nails both by conditioning them and by inactivating the organisms that might cause infections.

Use of cleansing light that is properly modified (in amplitude, pulse width, dosage, etc.) can result in the application of less total light dosage that would be require to clear some nails using germicidal light alone or conditioning light alone.

For example, in the clinical trials described earlier the application of 22 J/cm2 of UVC light at 254 nm (Prototype A) and the application of approximately 4.4 J/cm2 of light in the UVC range (Prototype B) had approximately the same effect with respect to new nail growth which was approximately 2.5 mm when measure 12 weeks after the first treatment. However, although Prototype B had only 20% of the UV of Prototype A, it was much more effective in improving the appearance of the nails. This is due to the synergistic action of germicidal and conditioning light. Thus cleansing light is a more efficient method to improve the appearance of nails than either germicidal light or conditioning light alone.

One of our discoveries is that nails that are not infected too can be treated using cleansing light so that their appearance can be improved. While not wishing to be bound by specific wavelengths, it appears that a substantial amount of cleansing light that can improve the appearance of nails is in the ultraviolet range of 240 nm to 400 nm though this range also extends into the UVC band and the visible spectrum. It should also be noted that hair can be bleached by sunlight that has no UVC and only about 1% as much UVB as UVA. Therefore, it is reasonable to expect that UVA light alone can be used to condition a nail and improve its appearance, and this use of UA light is as part of this invention.

Cleansing light can also be used in a similar manner to improve the appearance of skin. While sun tanning using UVA light is very common, other light spectrums have not been used to improve the appearance of skin and are thus expressly claimed as part of this invention.

### Other Notes Concerning Germicidal, Conditioning, and Cleansing Light

It should be noted that there are a variety of ways that light can be modified to make it more efficacious.

For example, other wavelengths of light can be used synergistically to bring about results superior to the use of narrow bandwidths.

Topical applications can also increase the efficacy of light treatment. One kind of topical application would be one that acted germicidally either by itself or combined with light. For example a fungicide or other antibiotic could be applied while using phototherapy or immediately thereafter and it should work synergistically with the light. This would be especially true if the application of light dehydrated the nail so that it would more readily absorb the antibiotic.

Another type of topical application would be one that acted to condition the nail or skin either by itself or combined with light. For example, a reducing bleach such a lemon juice or vinegar is often used to accelerate the bleaching of hair in sunlight. A similar compound could also be added to a nail or skin treated with conditioning light to bring about a similar effect.

The environment of the nail or skin could also be conditioned by the application of heat or cold, increasing or decreasing the pH of the nail or skin, increases or decreasing the oxygen surrounding the nail, etc. These environmental changes could stress any organisms that were in the nail and skin negatively affecting its appearance.

### Notes on Lamps

Germicidal, conditioning, and cleansing light can be provided by a variety of different kinds of lamps such as mercury, xenon, halogen, lasers, light emitting diodes, etc. Light can be modified so that it is continuous, pulsed, coherent, non-coherent, etc. These examples are intended to be representative and not exhaustive. Any device that can generate light in the proper wavelengths, modulations, and dosages can be used for germicidal, conditioning, and/or cleansing light. Preferably one device is capable of applying each of germicidal, conditioning and cleansing light, however more than one device may be used without departing from the scope of the invention. More preferably switches or automatic or manual settings are available on the device to selectively apply light that is germicidal, conditioning or cleansing light, depending on which one is needed. It is understood, that more finite settings within each of the categories may be necessary. Alternatively, the type of light may be set by selecting characteristics of the light to be applied to the patient that would have the effect of being germicidal, conditioning or cleansing light in the particular application. In a further embodiment, one light device may be used have at least three settings including germicidal light, conditioning light, and cleansing light, and a selector for selecting from each of these settings for applying the selected type of light to a patient.

Specifically, pulsed xenon light has many of the characteristics that make it an attractive germicidal, conditioning, and/or cleansing light. While the germicidal abilities of xenon lamps have been recognized for some time, its ability to condition the nail is both a novel and unobvious part of this invention

Several characteristics of xenon pulsed light make it attractive as both a germicidal light and a conditioning light and, therefore, a good cleansing light:
1. Pulsed xenon lights have a high percentage of UV light, emitting approximately 10% to 45% of its light below 400 nm.
2. Short, high power pulses appear to inactivate organisms better than the same amount of power spread continuously.
3. Short, high power pulses are more likely to break the chemical bonds of chromophores than the same amount of power spread continuously.
4. The pulsing of xenon (and other gasses) with short, high energy electrical pulses makes a higher percentage of the light emit in the UV range so that a xenon pulsed light operating under low current will emit approximately 12% to 20% of light in the UV range, whereas a xenon pulsed light operating in the high current mode will emit approximately 30% to 45% in the UV range. Intermediate operation between these two modes can bring the total pulsed spectrum possibilities such that pulsed xenon light can be between approximately 12% to 45% in the UV range. This compares with a regular xenon lamp UV output of approximately 10% or less.
5. Pulsing of the light permits a large dose of light to be applied in a very short actual application time. Many pulsed lights operate with pulses that are about 10 to 50 microseconds and are rarely pulsed more than 120 times per second. A 50 microsecond pulse that pulses 120 times is ‘on’ less than 1% of the time. Thus, not only can high doses be applied but there is also time for the surface being treated to cool between pulses which limits heat buildup. Though microsecond pulses are discussed in this application, the disclosed invention could make use of nano, pico, or femto-second pulses to enhance the efficacy of treatment.
6. Pulsed light is broad spectrum compared with monochromatic light such as that emitted by a low pressure mercury lamp, and the dosage required to obtain an improvement of the appearance of the nail can be applied in less time than the monochromatic light.
7. Pulsed light appears to penetrate better than continuous light. This may be due to two-photon absorption resulting in two longer wavelength photons penetrating more deeply than one shorter wavelength photon could penetrate and then combining to emit one photon of light of a shorter wavelength. This phenomena is well recorded but is a relatively unusual event. However, when a large number of photons are emitted over a short period of time this phenomena can occur and is the basis for two photon microscopy. In two photon microscopy a femtosecond laser is often used to maximize the production of two-photon light, however, it is possible to use a light with longer pulses. The longer pulses will decrease the yield of the two-photon light but the amount produced may still be of therapeutic value.

Two photon absorption (TPA) is the simultaneous absorption of two photons of identical or different frequencies in order to excite a molecule from its ground state to an excited state. For example two photons at 500 nm can be absorbed simultaneously and produce the same frequency (and effect) of a 250 nm photon. This phenomenon is not common statistically since both photons must arrive simultaneously, and other specialized conditions must be met. However, this phenomenon can be particularly important when applied to the generation and application of germicidal and conditioning light for the treatment of skin and nails since germicidal and conditioning light relies heavily on UV light which does not penetrate well.

For example, two photons at 500 nm might penetrate significantly deeper than a 250 nm photon and once they penetrate more deeply and are absorbed simultaneously then can then have a similar effect as a 250 nm photon. Thus, using two photon absorption light can penetrate more deeply and still have the germicidal effect of light of a much shorter wavelength. It should be noted that while two photon absorption is rare this is somewhat counterbalanced by the tremendous efficacy of a small amount of germicidal light to inactivate an organism. Additionally, pulsing of light increases the density of photons generated tremendously which also increase the probability of two photo absorption. Thus this phenomenon can be enhanced by progressively decreasing the pulse width and increasing the magnitude of the pulse. Therefore, two photon absorption is claimed as a specific mechanism of efficacy to improve the appearance of skin and nails particularly when pulsed light is used.

Other similar phenomena my also enhance the penetration and/or effectiveness of pulsed light. These method to increase penetration are specifically claimed as inventions in this application.

### Description Of Preferred Embodiments

In the preferred embodiment, cleansing light is applied to a nail to achieve both a germicidal and a conditioning effect that improves the appearance of the nail or skin. The cleansing light applied may be applied in several doses for convenience, to limit total UV exposure at any one session, to minimize discomfort, and/or to improve the efficacy of the treatment.

Total dosage for any one treatment is preferably approximately 310 J of light in the range of 180 nm to 880 nm with 50 J of this light in the UV range and approximately 4 to 5 J of that light in the UVC range. These ranges should be taken as approximate and may be varied from 50% to 200% or more depending on the actual condition to be treated. Thus total preferred treatment could be from approximately 150 J to 600 J per treatment in the 180 nm to 880 nm range with additional light in both higher and lower ranges (specifically 100 nm to more than 2600 nm) with approximately 25 J to 100 J of this light being in the UV range of 180 nm to 400 nm and specifically 2 J to 10 J of this light being in the narrow UVC range of 240 nm to 280 nm.

For improving the appearance of the skin the dosages listed above may be reduced by half or more as appropriate. Total number of treatments may be from 1 to 10 with 4 being the currently preferred embodiment.

The dosages of light above can be provided with a xenon pulsed light operating in a low current mode and generating a UVC dosage of 1 to 20 mw/cm2 at a distance of about 2-inches from the nail being treated. Continuous Lamp Operation versus Hi and Low Current Pulsed Light Operation. Lamps that operate continuously emit only a very small portion of their light in the UV region. For example, a xenon arc lamp operating continuously emits approximately 10.5% of its light in the UV region (2.0% in UVC, 4.0% in UVB, and 4.5% in UVA). This small amount of light in the UV range, particularly the low amount in the UVC range, makes the use of continuous spectrum light for germicidal use problematic.

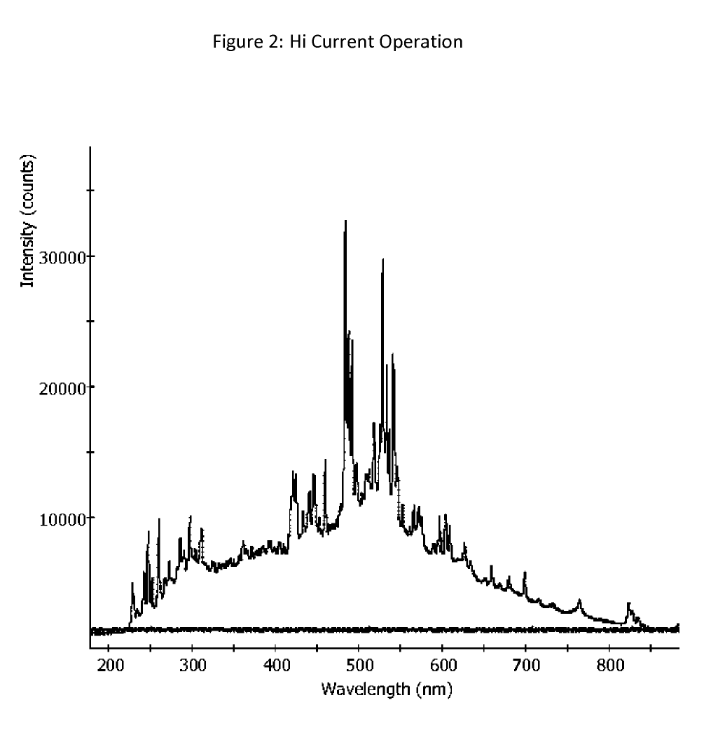

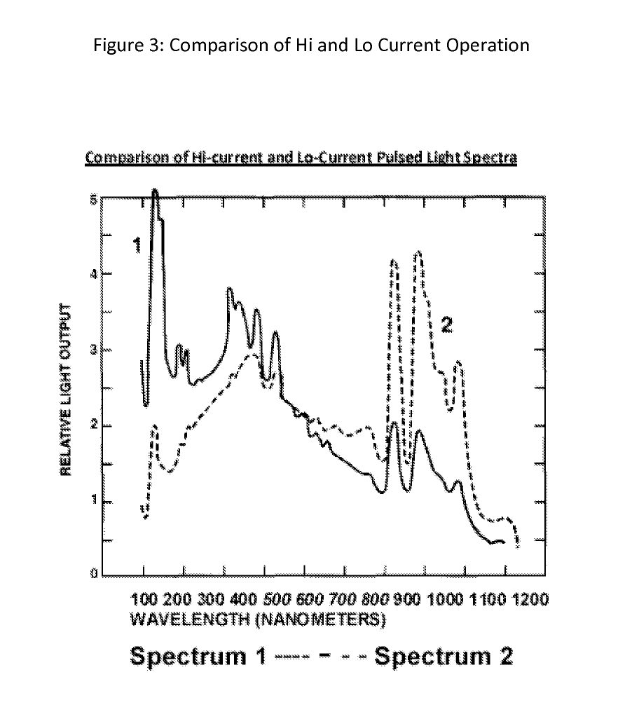

However, the amount of UV in the light spectrum can be significantly increased by pulsing of light. When light is pulsed a much higher instantaneous current is passed through the arc and the resulting light has a much higher percentage of high energy light (i.e. light in the UV range). Pulsing of light can increase the amount of UV in the light spectrum to up to 30% or more. The shift of light into the UV spectrum increases as the pulse dose increases. While the shift is somewhat continuous as the pulse dose increases (i.e. as the input dose is increased progressively more UV is produced with each pulse) two major pulse regimes have been identified and are called Hi (“High”) Current and Lo (“Low”) Current Operation. The boundary line between these two types of operation is not sharply distinct, however, the boundary line is roughly in the range of an input power dosage of between 4,000 amps/cm2 and 6,000 amps/cm2. For our purposes, we define high current operation as occurring at input power dosages of greater than 4,000 amps/cm2 (4 Kv/cm2). Low current operation (FIG. 1) can result in 20% or more of the light being produced in the UV range which is twice the UV production compared to continuous operation. High current operation (FIG. 2) can increase the percentage of UV to 30% or more and is especially efficient at creating a high percentage of UVC. FIG. 3 shows a comparison of the light spectra in the low and high current mode. Additionally, pulsing of the light can increase the overall conversion efficiency of input energy to light produced. Thus, pulsing can also increase the total amount of light compared to continuous operation.

Pulsed light is therefore especially useful for the generation of germicidal light. It is also extremely useful to break chromophores making them into smaller molecules which have less color, thus pulsed light is also useful to improve the appearance of skin and nails. Use of pulsed light to improve the appearance of skin and nails is a specific claimed invention of this application with Hi Current operation being the preferred mode of operation although Lo Current operation can also be used with somewhat less efficacy.

Another preferred embodiment would be to decrease the distance and even eliminate the distance so that the light is applied directly to the top of the nail. The intensity of light applied to an area being treated is directly related to the distance the light is from the area treated. For example if a light is placed 12-inches from an area being treated it the light reaching the area being treated will be only about 20% as intense as if the light was placed 2-inches from the area treated. Conversely, if the light was move to 1-in from the area being treated it would about 40% more intense than a light place 2-in from the area being treated. For most practicable purposes, therefore, treatment using light is contemplated using a light that is held less than 12-inches from the area being treated. However, in some cases the distance can be increased if measure are taken to concentrate the light by the use of mirrors, lenses, or other similar means. Unless otherwise noted in this application, the preferable distance for the application of light to receive the most effective treatment is within 1-2 inches from the surface of the nail or skin.

The pulsed light has high peak power pulses which help inactivate organisms and breach the chemical bonds of chromophores. If the UVC dosage is approximately 9 mw/cm2 the light can be applied for 8 minutes to achieve a UVC dosage of 4.4 J which is the current preferred embodiment. Other preferred embodiments can provide UVC dosages of between 2 to 10 J per session or more if pain is not experienced during treatment. In addition to the UVC applied, Far UV, UVB, UVA, and visible light should be applied in accordance with the operating characteristics of the xenon pulsed light described below.

The operating characteristics of the xenon pulsed light for the current preferred embodiment are as follows assuming an 8 minute application time (percent of light in the 180 nm to 880 nm range is included in parentheses):
Far UV from 180 nm to 240 nm—1.8 J/cm² (0.7%)
UVC from 240 nm to 280 nm—4.4 J/cm² (1.4%)
UVB from 280 nm to 320 nm—8.7 J/cm² (2.8%)
UVA from 320 nm to 400 nm—34.5 J/cm² (11.2%)
Visible light from 400 nm to 750 nm—260 J/cm² (83.0%)
Infrared from 750 nm to 880 nm—1 J/cm² (0.9%)
Infrared above 880 nm—estimated to be about 60 J/cm²

The preferred embodiment can be applied to infected or non-infected nails to improve their appearance.

This embodiment can also be applied to skin with the dosage being reduced by approximately 50% to 90% so that it is well tolerated by the skin. Substantial reduction of dosages can be used to beneficially treat the skin due to its thinness compared with nails. Reduction of dosage may also result in the treatment being better tolerated, e.g., to reduce burns. One type of improvement in skin appearance specifically claimed in this invention is lightening, bleaching or whitening of the skin.

In the preferred embodiment the time of treatment for nails is 8 minutes. In other preferred embodiments this time can vary from less than 1 second to 30 minutes. Treatment times for skin can be substantially less than for nails.

### Description of Further Preferred Embodiments

In another preferred embodiment cleansing light is applied using a xenon pulsed lamp in high current operation with the following characteristics (percent of light in the 180 nm to 880 nm range is included in parentheses):
Far UV from 180 nm to 240 nm—1.8 J/cm² (2.2%)
UVC from 240 nm to 280 nm—4.4 J/cm² (8.5%)
UVB from 280 nm to 320 nm—8.7 J/cm² (9.9%)
UVA from 320 nm to 400 nm—34.5 J/cm² (15.7%)
Visible light from 400 nm to 750 nm—260 J/cm² (61.9%)
Infrared from 750 nm to 880 nm—1 J/cm² (1.8%)

The light has a UVC component of approximately 9 mw/cm2 and is applied for approximately 8 minutes during each session. In this embodiment the power of the lamp can be increased and the application time decreased accordingly. In this embodiment the preferred number of sessions is 4 but can vary from 1 to 10 or more as needed to treat the nail. In this embodiment the total dosage of each session may be varied from 50% to 200% in accordance with the condition of the nail with clearer nails receiving a lower dose. In this embodiment the nail can be infected or non-infected. Treatment of the skin can be accomplished by this embodiment with the dosage being reduced accordingly.

### Further Preferred Embodiment

Another preferred embodiment is a device that can be varied to produce the conditions of the preferred embodiments. Specifically, the device has the capability of delivering the target dosages list herein and varying the dosage level as necessary to treat specific conditions as determined by the person who administers the treatment. The device is able to deliver high power pulsed light capable of either emitting germicidal, conditioning, or cleansing light.

### Further Preferred Embodiment

In another preferred embodiment the light can be applied using a xenon pulsed lamp to condition the nail or skin. The lamp and dosages applied can be in accordance with the dosages described in the preferred embodiments above with the difference that this treatment is applied primarily to condition and improve the appearance of the nails or nails and not to treat an infection.

### Further Preferred Embodiment

In another preferred embodiment the light can be applied to condition or cleanse a nail or skin using a lamp similar to those currently used to remove hair or tattoos, or those used to remove spider veins by modifying the light to permit bandwidths below approximately 400 nm to reach the area to be treated instead of being filtered out. The lamp and dosages applied can be in accordance with the dosages described in the preferred embodiments above with the difference that this treatment is applied primarily to condition and improve the appearance of the nails and skin and not to treat an infection.

### Further Preferred Embodiment

In another preferred embodiment the light can be applied to condition or cleanse a nail using a lamp similar to those currently used to treat psoriasis and other disorders, remove hair or tattoos, or those used to remove spider veins but modified so that light in the bandwidths below approximately 320 nm are allowed to reach the area to be treated instead of being filtered out. The lamp and dosages applied can be in accordance with the dosages described in the preferred embodiments above with the difference that this treatment is applied primarily to condition and improve the appearance of the nails and skin and not to treat an infection.

### Further Preferred Embodiment

In another preferred embodiment the light can be applied to condition or cleanse a nail using a lamp similar to those currently used to treat psoriasis and other disorders, remove hair or tattoos, or those used to remove spider veins but modified so that light in the bandwidths below approximately 280 nm are allowed to reach the area to be treated instead of being filtered out. The lamp and dosages applied can be in accordance with the dosages described in the preferred embodiments above with the difference that this treatment is applied primarily to condition and improve the appearance of the nails and skin and not to treat an infection.

### Further Preferred Embodiment

In another preferred embodiment the light can be applied using a xenon pulsed lamp to condition the nail. The lamp and dosages applied can be in accordance with the dosages described in the preferred embodiments with the exception of those between 280 nm and 320 nm. Wavelengths between 280 nm 320 nm may be filtered out to decrease erythema and pain associated with this bandwidth.

### Further Preferred Embodiment

In another preferred embodiment the light can be applied using a lamp to condition the nail or skin. The lamp and dosages applied can be in accordance with the dosages described in the preferred embodiments. All wavelengths below 320 nm may be filtered out since the primary purpose is to improve the appearance of the nail and skin and not treat an infection.

### Further Preferred Embodiment

In another preferred embodiment conditioning light is applied to improve the appearance of the nail and skin. In this embodiment the light applied has substantially all of its bandwidth in the UVA range (320 nm to 400 nm). In this embodiment the preferred dosage is approximately 30 to 40 J/cm2 of UVA per session but this may be varied from 15 to 80 J/cm2 or higher if the light can be applied safely.

### Further Preferred Embodiment

In another preferred embodiment the light is both germicidal and conditioning and the ratio between germicidal and conditioning light can be adjusted.

### Further Embodiment

Further embodiments include the varying of dosages applied, the number of sessions, what type of light is used, etc.

A further embodiment can use UVC only to condition and/or cleanse the nail and skin. In this case the UVC is used only to condition a nail or skin or to both condition the nail or skin and treat and infection. Dosages of UVC in this case could vary from 0.1 J/cm2 to 200 J/cm2 with lower dosages being more appropriate if means are used to increase the amount of light that can penetrate the nail an skin.

A further embodiment includes the means to adjust the pulses (their frequency, duration, amplitude, etc.) to enhance treatment.

A further embodiment is the use of no filter or a filter than is optically transparent in the UV range.

### Further Alternative Embodiments

A continuous xenon lamp can be used to provide the required light

A continuous or pulsed mercury lamp can be used to provide the required light

A continuous or pulsed mercury-xenon lamp can be used to provide the required light.

Other types of lamps such as lasers, LEDs, halogen, excimer, etc. can be used to provide the light necessary for treatment

Lemon juice, vinegar, sulfur dioxide, or other reducing agents can be applied to enhance treatment.

Fungicide or other antibiotics can be applied before or after treatment to enhance its efficacy.

### Summary, Ramifications, and Scope

Accordingly, this invention can be used to improve the appearance of nails and skin.
With respect to treatments that rely on inactivating the organism that causes nail and skin infections, the results of this treatment can be seen more quickly
Can improve the appearance of nails and skin that are not infected whether they were ever infected or whether they were infected but when the infection was treated they still remained unclear.
Compared with treatment by UVC alone this treatment requires a much lower UV dosage (both with respect to UVC alone and with UV in total). Thus treatment is safer and can be administered more quickly

Although the descriptions above contain many specificities, these should not be construed as limiting the scope of the invention but merely as providing illustrations of some of the presently preferred embodiments of this invention. For example, other sources of radiation may be used if they have the properties necessary to inactivate organisms, penetrate sufficiently, and are safe to humans or animals, etc. Each element and objective of the application may be used separately, together, or in various combinations, and the permutations provided above are for illustrative purposes and do not limit the invention to these examples. Thus the scope of this invention should be determined by the appended claims and their legal equivalents, rather than by the examples given. Where the term “or” is used, this can be the exclusive or non-exclusive “or” unless explicitly stated otherwise.

We claim:

1. A method of using germicidal light to prevent or treat nail or skin infections, comprising:
a. providing a light source emitting germicidal light;
b. applying the germicidal light to a nail or skin infection in discrete pulses at one second per pulse or less;
c. wherein at least 10% of the light from said germicidal light emitted in the range of 100 nm and 880 nm is emitted in the range of 100 and 400 nm; and
d. wherein said germicidal light is emitted from said light source in a light dosage selected from a group consisting of 15 J to 600 J of light in the range of 180 nm to 880 nm, 2.5 J to 100 J of light in the range of 180 nm to 400 nm, and 0.2 J to 10 J of light from 240 nm to 280 nm.

2. The method of using germicidal light of claim 1, wherein said germicidal light pulse widths are selected from the range of 1 femtosecond to 1000 nanoseconds.

3. The method of using germicidal light of claim 1, wherein said germicidal light pulse widths are selected from the range of 1 microsecond to 1000 milliseconds.

4. The method of using germicidal light of claim 1, wherein said germicidal light is emitted from said light source in a light dosage selected from a group consisting of 150 J to 600 J of light in the range of 180 nm to 880 nm, 25 J to 100 J of light in the range of 180 nm to 400 nm, and 2 J to 10 J of light from 240 nm to 280 nm.

5. The method of using germicidal light of claim 1, wherein said germicidal light is emitted from said light source in a light dosage selected from a group consisting of 15 J to 300 J of light in the range of 180 nm to 880 nm, 2.5 J to 50 J of light in the range of 180 nm to 400 nm, and 0.2 J to 5 J of light from 240 nm to 280 nm.

6. A method of using conditioning light to improve the appearance of nails or skin, comprising the steps of:
a. examining the nail or skin or a user;
i. providing a light source emitting conditioning light;
ii. applying the conditioning light to a nail or skin infection in discrete pulses of light;
iii. wherein at least 10% of the light from said conditioning light emitted in the range of 100 nm and 880 nm is emitted in the range of 100 and 400 nm; and
iv. wherein such light has a light dosage selected from a group consisting of 15 J to 600 J of light in the range of 180 nm to 880 nm, 2.5 J to 100 J of light in the range of 180 nm to 400 nm, and 0.2 J to 10 J of light from 240 nm to 280 nm

7. The method of using conditioning light of claim 6, wherein said conditioning light pulse widths are selected from the range of 1 femtosecond to 1000 milliseconds.

8. The method of using conditioning light of claim 6, wherein said conditioning light is emitted from said light source in a light dosage selected from a group consisting of 150 J to 600 J of light in the range of 180 nm to 880 nm, 25 J to 100 J of light in the range of 180 nm to 400 nm, and 2 J to 10 J of light from 240 nm to 280 nm.

9. The method of using conditioning light of claim 6, wherein such light has a light dosage selected from a group consisting of 1.50 J to 300 J of light in the range of 180 nm to 880 nm, 2.5 J to 50 J of light in the range of 180 nm to 400 nm, and 0.2 J to 5 J of light from 240 nm to 280 nm

10. The method of using conditioning light of claim 6, further wherein said provided light comprises a sufficient amount of light above approximately 320 nm capable of improving the appearance of the nail and skin.

11. A method of using cleansing light to treat and prevent infection and improve the appearance of nails or skin, comprising the steps of:
a. examining the nail or skin of a user;
b. only upon determination that the skin or nail of the user in an area of the skin or nail to be treated is discolored and infected:
i. providing a pulsed light source emitting cleansing light;
ii. applying the cleansing light to a nail or skin infection;
iii. Wherein at least 10% of the light from said cleansing light emitted in the range of 100 nm and 880 nm is emitted in the range of 100 and 400 nm; and
iv. Wherein such light has a light dosage selected from a group consisting of 15 J to 600 J of light in the range of 180 nm to 880 nm, 2.5 J to 100 J of light in the range of 180 nm to 400 nm, and 0.2 J to 10 J of light from 240 nm to 280 nm.

12. The method of using cleansing light of claim 11, wherein said cleansing light pulses have a width are selected from the range of 1 femtosecond to 1000 nanoseconds.

13. The method of using cleansing light of claim 11, wherein said cleansing light pulses have a width selected from the range of 1 microsecond to 1000 milliseconds.

14. The method of using cleansing light of claim 11, wherein said cleansing light is emitted from said light source in a light dosage selected from a group consisting of 150 J to 600 J of light in the range of 180 nm to 880 nm, 25 J to 100 J of light in the range of 180 nm to 400 nm, and 2 J to 10 J of light from 240 nm to 280 nm.

15. The method of using cleansing light of claim 11, wherein said cleansing light is emitted from said light source in a light dosage selected from a group consisting of 15 J to 300 J of light in the range of 180 nm to 880 nm, 2.5 J to 50 J of light in the range of 180 nm to 400 nm, and 0.2 J to 5 J of light from 240 nm to 280 nm.

16. A method of treating nails or skin of a subject comprising the steps of:
a. examining the skin or nail for infection of the subject;
b. providing means a light system for applying light to the subject, wherein the light system is configured to selectively provide germicidal light, conditioning light, and cleansing light;
c. using the light system to applying one of germicidal light, conditioning light or cleansing light to the subject's nail or skin.

17. The method of claim 16, further comprising:
treating the skin or nail of a user with a composition prior to applying the light to the skin or nail of the user, wherein the composition acts synergistically with the light to improve the appearance of the nail and skin;
said composition being selected from a group including fungicide, reducing bleach, addition of a compound to increase or decrease oxygen, addition of a compound to increase or decrease the temperature, addition of a compound to increase or decrease the pH of a nail and addition of a compound to increase the free radicals or singlet oxygen or otherwise alter the nail and skin in a beneficial manner.

18. The method of treating nails or skin of claim 16, wherein the light system is contained in one housing and is operated by a common power source within the housing.

19. The method of claim 16, wherein the light can be operated in a low current mode having a first light characteristic and a high current mode having a second light characteristic, wherein said light emits more UV light in said high current mode than said low current mode.

20. A method of increasing the translucency of nails of using light, comprising:
a. applying light at 9 mw/cm² for a total of at least 8 minutes to a nail less than 4 inches from said light, wherein at least 12.5 J/cm² of light is in the UV range;
b. wherein at least a portion of the nails are cleared by application of the applied light.

21. The method claim 20, wherein the at least 32 minutes of light is applied in at least four sessions.

22. The method claim 20, wherein the applied light energy breaks bonds of chromophores in the nails, thereby changing the appearance of at least a portion the nails to a lighter color or to increase its translucency.

23. A method of increasing the appearance of an area of skin or nail using light, comprising:
a. examining the area of skin or nail to determine if the area of skin or nail is discolored;
b. examining the area of skin or nail to determine if the area of skin or nail is infected;
c. providing a light source for selectively applying each of germicidal light, conditioning light and cleansing light;
d. upon determination that the area of skin or nail is infected and not discolored, applying germicidal light to the area of skin or nail;
e. upon determination that the area of skin or nail is discolored and not infected, applying conditioning light to the area of skin or nail; and
f. upon determination that the area of skin or nail is discolored and infected, applying cleansing light to the area of skin or nail.
